(19) United States
(12) Patent Application Publication
(10) Pub. No.: US 2005/0143476 A1
(43) Pub. Date: Jun. 30, 2005

(54) Emulsion composite

(76) Inventors: Masahiro Okajima, Aichi (JP); Fumiyoshi Ishii, Tokyo (JP); Kenji Sugibayashi, Saitama (JP)

Correspondence Address: LADAS & PARRY, Suite 2100, 5670 Wilshire Boulevard, Los Angeles, CA, 90036-5679, US

(21) Appl. No.: 10/803,469

(22) Filed: Mar. 17, 2004

(30) Foreign Application Priority Data
Nov. 7, 2003 (JP) 2003-378756

(51) Int. Cl. B01F 3/08; A61K 7/46

(52) U.S. Cl. 516/53; 512/1

(57) ABSTRACT

The invention aims to provide an emulsion composite which is excellent in safety and stability and which can easily be produced. 20 g of soybean oil and 2.4 g of glycerin were mixed by being stirred with a magnetic stirrer. The mixture was added to strong alkali ionic water to adjust the total amount to 200 g. The solution was subjected to primary emulsification in a boiling water bath at 12,765 rpm for a stirring time of 30 minutes using an autohomomixer. Necessary amounts of strong alkali ionic water and glycerin were then added such that the volume and the osmotic pressure became predetermined values. This sample was subjected to secondary emulsification at a pressure of 1,000 bar with 40 pass times using a high-pressure homogenizer to prepare an O/W emulsion (emulsion composite).

### BACKGROUND OF THE INVENTION

1. Field of the Invention

The present invention relates to an emulsion composite which is used to produce drugs or beauty products.

2. Description of the Related Art

An emulsion composite has been so far incorporated in drugs or beauty products. This emulsion composite is obtained by emulsifying water and oil with a nonionic surfactant such as purified yolk lecithin or soybean lecithin.

In case of drugs which are intravenously administered, a particle diameter of oil drops of the emulsion composite is usually adjusted to less than 220 nm. This is because when the mean particle diameter exceeds 500 nm, side effects such as fat embolism, thrombophlebitis and deep venous thrombosis might occur in using the emulsion composite in drugs which are intravenously administered.

A surfactant which is used in dispersing oil drops has a high permeability into a biological membrane. Thus, when the amount of the surfactant is large, the surfactant has qualities that it dissolves the biological membrane, enters blood vessels from the skin and circulates throughout the body to cause hemolysis and it interacts with proteins to cause denaturation. Accordingly, an emulsion composite containing a large amount of the surfactant has posed a problem of safety.

Further, the ordinary emulsion composite has been problematic in that oil drop particles are agglomerated over the course of time after production and it is difficult to store the emulsion composite for a long period of time. Moreover, a process for production of the emulsion composite requires the long-term treatment with a homogenizer or the like for reducing a particle diameter of oil drops. Thus, there has been a problem that the production takes much time and labor.

No document of the related art has been found on the invention.

### SUMMARY OF THE INVENTION

Under these circumstances, the invention has been made, and it aims to provide an emulsion composite which is excellent in safety and stability and which can easily be produced.

The invention is to provide an emulsion composite comprising strong alkali ionic water as a dispersion medium and oil drop particles made of an oil component. Since the dispersion medium is strong alkali ionic water in the emulsion composite of the invention, the dispersibility of oil drop particles is high. Thus, a surfactant is not incorporated, or an amount of a surfactant can be reduced.

Accordingly, the emulsion composite of the invention is free from the following problems which are defects encountered by incorporating a large amount of a surfactant. That is, the surfactant dissolves the biological membrane, enters blood vessels from the skin and circulates throughout the body to cause hemolysis, and it interacts with proteins to cause denaturation. Further, since the emulsion composite of the invention is excellent in stability of oil drop particles after emulsification, the oil drop particles are not agglomerated over a long period of time after emulsification. For this reason, for example, drugs or beauty products produced using the emulsion composite of the invention can stably be stored over a long period of time.

The emulsion composite of the invention can easily be reduced in particle diameter (for example, less than 200 nm) of the oil drop particles in the process for producing the same. Consequently, the emulsion composite of the invention is easy to produce. Further, the diameter of the oil drop particles can be reduced in the emulsion composite of the invention. Accordingly, even when drugs using this emulsion composite are intravenously administered, there is no likelihood that side effects such as fat embolism, thrombophlebitis and deep venous thrombosis occur.

The strong alkali ionic water is physically electron-excessive water obtained by electrolyzing natural water, passing electricity through a special diaphragm device and pressurizing the resulting water. As the strong alkali ionic water, S-100 (trade name for a product manufactured by K.K. A. I. System Product, Japan) is exemplified. Examples of the oil component include soybean oil, olive oil, jojoba oil, sunflower oil and the like.

It is preferable that the mean diameter of the oil drop particles is, for example, 200 nm or less. The reason is that when the composition is used in drugs which are intravenously administered, there is no likelihood that side effects such as fat embolism, thrombophlebitis and deep venous thrombosis occur.

The emulsion composite of the invention is further characterized in that the strong alkali ionic water is super-reductive water. Since the strong alkali ionic water is super-reductive water, the emulsion composite of the invention is far superior in dispersibility of the oil drop particles, stability after emulsification and ease of reduction in diameter of oil drop particles.

The super-reductive water refers to electrolytic water having a hydrogen ion concentration of pH 12 or more and an oxidation reduction potential (ORP) is 0 mV or less and ionic water having an osmotic pressure of 100 (mOsm) or less. As the super-reductive water, for example, S-100 (trade name for a product manufactured by K.K. A. I. System Product, Japan) is exemplified.

The emulsion composite of the invention has pH from 8 to 11. Since the pH is from 8 to 11, the emulsion composite is far superior in dispersibility of the oil drop particles, stability after emulsification and ease of reduction in diameter of oil drop particles.

### BRIEF DESCRIPTION OF THE DRAWINGS

FIG. 1 is a graph showing the results of measuring the stability of emulsification for making sure the effect of the emulsion composite of the invention;

FIG. 2 is a graph showing the results of measuring the mean particle diameter for making sure the effect of the emulsion composite of the invention;

FIG. 3 is a graph showing the results of measuring the height of separate phase for making sure the effect of the emulsion composite of the invention;

FIG. 4 is a graph showing the results of measuring the height of separate phase for making sure the effect of the emulsion composite of the invention; and

FIG. 5 is a graph showing the results of measuring the surface tension for making sure the effect of the emulsion composite of the invention.

### DESCRIPTION OF THE PREFERRED EMBODIMENTS

Embodiments of the emulsion composite of the invention are described below.

(A) First, emulsion composites of EXAMPLE 1 were produced as follows.

### EXAMPLE 1-1

First, 20 g of soybean oil and 2.4 g of glycerin were mixed by being stirred with a magnetic stirrer (HS-4SP, manufactured by iuchi). The mixture was added to S-100 (trade name for a product manufactured by K.K. A. I. System Product) which is strong alkali ionic water and super-reductive water to adjust the total amount to 200 g. The solution was subjected to primary emulsification in a boiling water bath at 12,765 rpm for a stirring time of 30 minutes using T. K. Autohomomixer TYPE. M (trade name for a machine manufactured by Tokushu Kika Kogyo K.K.).

After the primary emulsification was completed, necessary amounts of strong alkali ionic water (S-100) and glycerin were added such that the volume became 200 ml and the osmotic pressure became 278 mOsm.

Further, the sample was subjected to secondary emulsification at a pressure of 1,000 bar with 40 pass times using a high-pressure homogenizer (GEO Niro Soavi S.p. A Via M. da Erva Edoari, 29A/A-43100 PARMA ITALY TYPE NS1001L2K) to prepare an O/W emulsion (emulsion composite).

### EXAMPLE 1-2

First, 2.4 g of lecithin (surfactant) was completely dissolved in a small amount of ethanol, and 20 g of soybean oil and 2.4 g of glycerin were then added. The mixture was uniformly stirred with a magnetic stirrer. Ethanol was then removed with an evaporator (BUCHI Vacuum Controller B-720).

The mixed sample was then added to 175.2 g of strong alkali ionic water to adjust the total amount to 200 g. Primary emulsification was conducted in a boiling water bath at 12,765 rpm for a stirring time of 30 minutes using T. K. Autohomomixer. After the primary emulsification was completed, a necessary amount of strong alkali ionic water (S-100) was added such that the volume became 200 ml.

Further, the sample was subjected to secondary emulsification at a pressure of 1,000 bar with 40 pass times using a high-pressure homogenizer to prepare an O/W emulsion (emulsion composite).

In EXAMPLE 1, the following emulsion composite was prepared as a comparative example.

### COMPARATIVE EXAMPLE 1

First, 2.4 g of lecithin (surfactant) was completely dissolved in a small amount of ethanol, and 20 g of soybean oil and 2.4 g of glycerin were then added. The mixture was uniformly stirred with a magnetic stirrer. Ethanol was then removed with an evaporator (BUCHI Vacuum Controller B-720).

The mixed sample was then added to 172.6 g of distilled water to adjust the total amount to 200 g. Primary emulsification was conducted in a boiling water bath at 12,765 rpm for a stirring time of 30 minutes using T. K. Autohomomixer.

After the primary emulsification was completed, a necessary amount of a 2.5% glycerin aqueous solution was added such that the volume became 200 ml. Further, the sample was subjected to secondary emulsification at a pressure of 1,000 bar with 40 pass times using a high-pressure homogenizer to prepare an O/W emulsion (emulsion composite).

(B) The effects brought forth by the emulsion composites of EXAMPLE 1 are described below.

(i) Since the emulsion composite of EXAMPLE 1-1 is free of a surfactant, drugs or beauty products containing the emulsion composite of EXAMPLE 1-1 do not involve the following problems which are defects encountered by incorporating a large amount of the surfactant. That is, the surfactant dissolves the biological membrane, enters blood vessels from the skin and circulates throughout the body to cause hemolysis, and it interacts with proteins to cause denaturation.

(ii) Since the emulsion composites of EXAMPLE 1 are excellent in stability after emulsification, the oil drop particles are not agglomerated over a long period of time after emulsification. For this reason, drugs or beauty products produced using the emulsion composites of EXAMPLE 1 can stably be stored over a long period of time.

(iii) The emulsion composites of EXAMPLE 1 can easily be reduced in particle diameter of the oil drop particles in the process for producing the same. Accordingly, the emulsion composites of EXAMPLE 1 are easy to produce. Since the diameter of the oil drop particles can be reduced, there is no likelihood that side effects such as fat embolism, thrombophlebitis and deep venous thrombosis occur even when intravenously administering drugs produced using the emulsion composites of EXAMPLE 1.

(iv) The emulsion composites of EXAMPLE 1 are stable because they hardly cause agglomeration of oil drops especially in the pH range of from 10.5 to 9. Accordingly, even when drugs or beauty products in this pH range are produced using the emulsions of EXAMPLE 1, the oil drop particles can stably be stored over a long period of time without agglomeration.

(C) Tests performed for making sure the effects brought forth by the emulsion composites of EXAMPLE 1 are described below.

(i) Test for stability of emulsification

Regarding EXAMPLES 1-1 and 1-2 and COMPARATIVE EXAMPLE 1, the samples after the primary emulsification and the volume adjustment were collected in color comparison tubes, and allowed to stand. A height of a separate phase that was formed by the agglomeration of oil drops was recorded every day from immediately after allowing to stand. The sample after the primary emulsification was used because the sample after the secondary emulsification was quite a high stability and it was difficult to evaluate the stability for a short period of time.

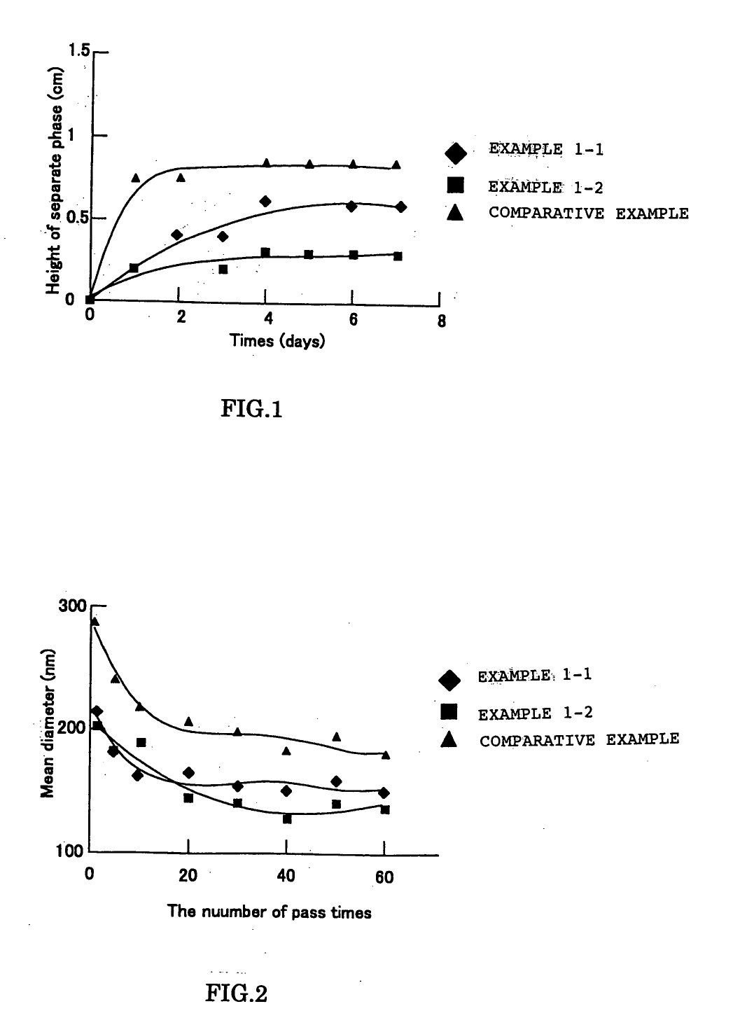

The experimental results are shown in FIG. 1. In the sample of EXAMPLE 1-2, it was most difficult to form the separate phase. The rate of formation of the separate phase at the initial stage (until day 1) was 8.3×10⁻³ cm/hr, and the height of the separate phase from day 4 was approximately 0.3 cm and constant.

In the sample of EXAMPLE 1-1, it was difficult to form the separate phase, next to the sample of EXAMPLE 1-2. The rate of formation of the separate phase at the initial stage (until day 1) was 8.3×10⁻³ cm/hr, and the height of the separate phase on day 7 was 0.6 cm and constant.

Meanwhile, in the sample of COMPARATIVE EXAMPLE 1, the separate phase was liable to form. The rate of formation of the separate phase at the initial stage (until day 1) was 31.3×10⁻³ cm/hr, and the height of the separate phase from day 4 was approximately 0.85 cm and constant.

Thus, the medical emulsions of EXAMPLES 1-1 and 1-2 were found to be preferably excellent in stability of emulsification.

(ii) Test for diameter of emulsion oil drop particles

Regarding EXAMPLES 1-1 and 1-2 and COMPARATIVE EXAMPLE 1, small amounts of the respective samples were collected when the numbers of pass times of the high-pressure homogenizer in the secondary emulsification were 1, 5, 10, 20, 30 and 40. Moreover, other samples were formed in the same manner as in EXAMPLES 1-1 and 1-2 and COMPARATIVE EXAMPLE 1 except that the number of pass times of the high-pressure homogenizer in the secondary emulsification were 50 and 60.

Then, the particle diameter of oil drops in the samples collected was measured using a submicron analyzer (NICMP 370/Autodilute Submicron Particles Sizer). The results are shown in FIG. 2.

In the emulsion composites of EXAMPLES 1-1 and 1-2, the particle diameters are already 210 nm and 200 nm when the number of pass times is 1, and reach 220 nm which is a mean particle diameter of a usual fat emulsion. The mean particle diameter is abruptly reduced until the number of pass times reaches 20, and is moderately reduced until the number of pass times reaches 40. When the number of pass times is 40, the mean particle diameters of the medical emulsions of EXAMPLES 1-1 and 1-2 were 149.3 nm and 127.7 nm respectively.

Meanwhile, when the number of pass times was 1, the particle diameter of oil drops in the emulsion composite of COMPARATIVE EXAMPLE 1 was 282.6 nm which was larger than 220 nm, the mean particle diameter of the usual fat emulsion. Thereafter, the mean particle diameter was reduced as the number of pass times was increased. When the number of pass times was 20, the mean particle diameter was 202.6 nm which was smaller than 220 nm, the mean particle diameter of the usual fat emulsion. As the number of pass times was increased to 40 or 60, the mean particle diameter was 181.2 nm or 178.4 nm respectively.

Thus, the emulsion composites of EXAMPLES 1-1 and 1-2 were found to be preferable because even through the number of pass times of the homogenizer in the emulsification was small, the particle diameter of oil drop particles could be reduced to make easy the production of the emulsion composites.

(iii) Test for influence of pH on a stability of emulsion composites

(a) Preparation of Samples

A necessary amount of a 1 mol/L acetic acid aqueous solution was added to the emulsion composite of EXAMPLE 1-1 such that pH indicated by a pH meter (F-22, trade name for a unit manufactured by Horiba) became 6 to prepare a sample having pH 6. Samples having pH 7, 8 and 9 were prepared in the same manner.

(b) Measurement of a Height of a Separate Phase

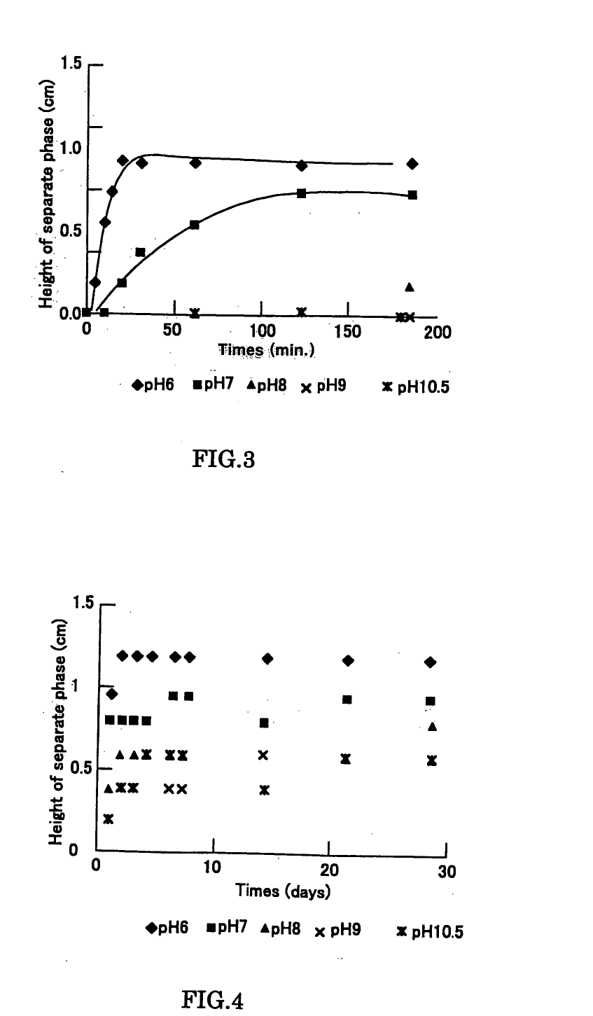

With respect to the samples prepared such that the predetermined pH became 6, 7, 8 and 9 as described above and a sample (pH=10.5) free of the acetic acid aqueous solution, the stability was evaluated from the height of the separate phase formed when the samples were charged into color comparison tubes and allowed to stand as in (i). The results are shown in FIGS. 3 and 4. FIG. 3 shows the results of evaluation for a short period of time from the start-up of the test till the lapse of 180 minutes, and FIG. 4 shows the results of evaluation for a long period of time from the start-up of the test till the lapse of 28 days.

In the stability for a short period of time from the start-up of the test, almost no formation of the separate phase caused by agglomeration of oil drops was observed in the sample with pH 8 or more as shown in FIG. 3. In the sample with pH 7, the height of the separate phase after 20 minutes was 0.19 cm, the rate of separation at the initial stage was 0.57 cm/hr, and the height of the separate phase after 120 minutes was 0.75 cm and constant. In the sample with pH 6, the height of the separate phase after 15 minutes was 0.75 cm, the rate of separation at the initial stage was 3.0 cm/hr, and the height of the separate phase after 20 minutes was 0.94 cm and constant.

In the stability over a long period of time after the start-up of the test, it could be confirmed, as shown in FIG. 4, that as the pH was increased, the height of the separate phase was decreased and stable.

(c) Measurement of a Surface Tension

With respect to the samples prepared such that the predetermined pH became 6, 7, 8 and 9 in (a) and the sample (pH=10.5) free of the acetic acid aqueous solution, the surface tension was measured.

The more the agglomeration of oil drop particles proceeds, the more the surface tension is decreased. Accordingly, the surface tension can be used as an index of the stability of the emulsion composite. The reason is as follows. The surface tension is increased between water-water molecules showing the same properties, and is decreased between water-oil molecules showing different properties. When the agglomeration of oil drop molecules proceeds, the area occupied by oil on the surface of the sample is increased, and the surface tension is decreased to increase the influence of an adhesion force between oil-water molecules.

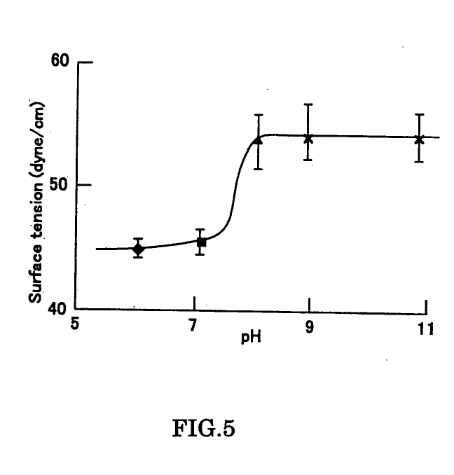

In the measurement of the surface tension, a du Nouy tensiometer was used, and the amount of the sample was set at 20 ml. The measurement was conducted five times for one sample, and the mean value was employed. The results of measurement are shown in FIG. 5. Since the samples with pH 8 or more showed the high surface tension of 55.4 dyne/cm, it was considered that agglomeration of oil drops did not occur. The surface tension of the sample with pH 7 was 44.7 dyne/cm.

Thus, the emulsion composite of EXAMPLE 1-1 was found to be preferable because of the high stability in the pH range of from 8 to 10.5.

The emulsion composite of the invention can also be used in beauty products or scents by containing a perfume therein. As the perfume, various known perfumes such as water-soluble perfumes and oil perfumes are available. These may be used either singly or in combination.

The invention is not limited at all to these Examples. It goes without saying that the invention can be performed in various embodiments without departing from the spirit and scope of the invention.

1. An emulsion composite comprising strong alkali ionic water as a dispersion medium and emulsion particles made of an oil component.

2. The emulsion composite as claimed in claim 1 or 2, wherein the strong alkali ionic water is super-reductive water.

3. The emulsion composite as claimed in claim 1, wherein pH is from 8 to 11.

4. The emulsion composite as claimed in claim 1, which further comprises a perfume.
